(19) United States
(12) Patent Application Publication
(10) Pub. No.: US 2017/0306369 A1
(43) Pub. Date: Oct. 26, 2017

(54) Method for Surfactant enhanced Enzymatic Hydrolysis

(71) Applicant: Maxwell Robert Ylitalo, Stillwater, MN (US)

(72) Inventor: Maxwell Robert Ylitalo, Stillwater, MN (US)

(21) Appl. No.: 15/138,291

(22) Filed: Apr. 26, 2016

(51) Int. Cl. C12P 19/02; C12P 19/14

(52) U.S. Cl. CPC C12P 19/02; C12P 19/14; C12P 2201/00

(57) ABSTRACT

A method for producing fermentable sugars from paper is described. The method comprises the steps of preparing an aqueous paper slurry, treating the paper slurry with non-ionic surfactant, adding an enzyme blend to the mixture and incubating the mixture at a temperature ranging from 30° C. to 50° C. to provide fermentable sugars for bioethanol production. The enzyme blend was optimized by combining three parts of cellulase and one part of cellobiase enzymes. The addition of non-ionic surfactant further improved the process yield where the optimum surfactant concentration at twice its critical micelle concentration was selected.

### FIELD OF THE DISCLOSURE

The present invention describes a method for converting paper, more specifically waste paper and recycled paper into fermentable sugars using enzymes and surfactants.

### BACKGROUND

Ethanol biofuel is considered a renewable fuel and it is used primarily in gasoline. Traditionally bioethanol has been derived from corn driving up the cost of food and taking up agricultural land that otherwise can be used for crops or animal feed. Therefore, attention has been directed at biomass as an alternative feedstock for making bioethanol.

Cellulose is the major component of plants and the most abundant organic molecule on earth. It is a polymer of sugar molecules which store energy from the sun. Cellulosic biomass is an attractive source of bioethanol because it is more cost effective compared to starch-based bioethanol (for example from corn) or sugar-based bioethanol (for example from sugar cane). One source of biomass is lignocellulose, which is readily available from agricultural and forestry wastes and from waste paper.

Cellulose can be converted into fermentable sugars by enzymatic hydrolysis, where the resulting sugars are fermented to make ethanol. Cellulase enzyme, typically derived from cellulose-degrading fungi, is currently used to hydrolyze biomass. However, converting biomass into fermentable sugars requires a pretreatment step in order to remove the lignin component in the biomass and to make cellulose fibers accessible to the enzyme. The pretreatment step is costly and energy intensive reducing the economic benefits of cellulosic ethanol.

Therefore, there is a need for a cost effective method to convert cellulose to fermentable sugar.

### SUMMARY

The present invention is directed to a novel method for producing fermentable sugars. The method comprises the steps of mixing an aqueous paper composition with non-ionic surfactant, adding enzyme to the paper blend, incubating the mixture to hydrolyze the paper fibers into fermentable sugars. After the incubation step, the mixture is filtered to separate the paper residue from the fermentable sugars solution. The enzymes used in this method comprise cellulase and cellobiase.

In a preferred embodiment the enzyme blend comprises three parts of cellulase to one part cellobiase. The enzyme blend concentration is less than 20% by weight of the paper and the surfactant concentration is at least twice its critical micelle concentration.

In one aspect of the inventive method, the enzyme blend is added to the paper/surfactant mixture at least one hour after adding the surfactant to the paper.

In a preferred embodiment the pH of the paper solution after adding the surfactant and enzyme blend is adjusted to be between 5.5 and 6.5.

In a second aspect of this invention an alternate method to produce fermentable sugars from paper is described. The alternate method comprises the steps of mixing paper with a buffer solution using high sheer mixing method. A non-ionic surfactant is added to the paper mixture followed by the addition of a blend of hydrolyzing enzymes. The mixture is incubated at elevated temperature to convert the paper to hydrolyzing sugars. The mixture is filtered to separate the paper residue from the fermentable sugars.

The above summary of the present invention is not intended to describe each disclosed embodiment or every implementation of the present invention. The Detailed Description and Examples that follow more particularly exemplify these embodiments.

### Advantages of the Invention

This invention provides a method to efficiently convert waste paper into fermentable sugars for bioethanol production. The method is based on using a unique combination of non-ionic surfactant and hydrolyzing enzymes that results in improved sugar yield.

### Definitions

As used herein, the term “paper” or “waste paper” describe a mixture of white office paper, newsprint paper, magazine pages, cardboard and any mixture of.

As used herein, the term “paper slurry” describes a mixture of paper in an aqueous solution where the solution is processed to break down the paper into uniform mixture in solution.

As used herein, the term “fermentable sugars” refers to all sugars and their mixtures that are water soluble and can be used as carbon source for microorganisms such as yeast to generate alcohol containing compounds.

As used herein, the term “critical micelle concentration” abbreviated as CMC refers to the surfactant concentration in water at ambient conditions above which micelles form.

### DETAILED DESCRIPTION

The present invention relates to a process to produce fermentable sugars from paper, more specifically from waste paper and recycled paper.

Cellulosic ethanol is commercially produced in many places in the world especially in Europe and the USA, and several companies are actively developing enzymes for cellulose hydrolysis.

Fungi degrade cellulose in nature by producing enzymes that hydrolyze cellulose, converting it to sugar. Those enzymes are typically a blend of endo-acting and exo-acting enzymes that work in synergy for biomass degradation. Therefore, enzymes derived from fungi can be used to hydrolyze cellulose fibers. Commercially available cellulase enzyme is a blend of three different enzymes: exo-cellulase, which breaks down inter polymer bonds; endo-cellulase, which breaks down intra polymer bonds; and cellobiase, which breaks down sugar dimer molecules. However, the cellulase blends used in biomass hydrolysis result in high amounts of cellobiose indicating insufficient cellobiase in the blend. Therefore, additional amount of cellobiase is needed for efficient hydrolysis of cellulose.

One of the embodiments of this invention relates to the blend of enzymes optimized to generate maximum yield of fermentable sugars. The blend comprises a mixture of cellulase and cellobiase enzymes where it was found from experiments that the optimum blend is three parts cellulase and one part cellobiase.

Cellulose is a linear polymer of D-glucose units linked together by glucosidic bonds. Hydrogen bonding between cellulose molecules results in the formation of highly ordered crystalline regions that are not readily accessible to hydrolyzing enzymes. Acid induced breakdown of the hydrogen bonds facilitates the hydrolysis of cellulose polymers.

The process of converting biomass to cellulosic ethanol has three main steps:
1) Pretreatment: to break down lignin and remove hemicellulose in order to make cellulose fibers accessible to hydrolyzing enzymes. This step is the most expensive and energy intensive step in the process.
2) Hydrolysis: to break down the cellulose into sugar. This reaction is done in acidic conditions or it is catalyzed by enzymes. Enzyme hydrolysis is usually preferred because its mild processing conditions do not require expensive reaction equipment or extensive energy.
3) Fermentation: where the resulting sugar is converted into ethanol by yeast fermentation.

In this invention the energy intensive pretreatment step is avoided by using waste paper and recycled paper as the hydrolysis feedstock. Therefore, the process of the current invention is more cost effective and sustainable than the methods described in conventional biomass hydrolysis.

Waste paper has the highest amount of cellulose compared to other biomass sources with office paper, containing up to 99% cellulose with no lignin component. Mixed waste paper in general contains about 70% cellulose. Therefore, paper is an attractive feedstock option for cellulosic ethanol production. It offers two key benefits: eliminates the costly pretreatment step to remove lignin and it is outside the human food chain.

It is known that cellulase enzymes can be used to hydrolyze cellulose into fermentable sugar. However, such an enzyme is costly and the loss of enzyme activity is one of the main limitations of cellulosic ethanol production. Therefore, there is a need to improve cellulase enzyme efficiency to lower the total amount of enzyme required for hydrolysis and reduce the cost of the process.

Adding surfactants may improve enzyme effectiveness because surfactants are known as good wetting agents in aqueous solutions, and non-ionic surfactants are known as good wetting agents for cotton fibers which are made of cellulose. Therefore, wetting the paper fibers with surfactant before initiating enzymatic hydrolysis is expected to improve the enzyme action by facilitating enzyme desorption.

Surfactants are organic compounds comprising hydrophobic “tail” and hydrophilic “head”. In general, the tail is made of hydrocarbon moiety, while the head almost always has an ionic charge. There are four main types of surfactants based on the type of ionic charge. Cationic surfactants have a positively charged head, anionic surfactants have a negatively charged head, non-ionic surfactants do not carry a permanent charge and zwitterionic surfactants (also known as amphoteric) carry both positive and negative charges on each molecule. When dissolved in aqueous solution, surfactants aggregate at the air/solution interface with the hydrophobic tail positioned outside the solution. As the concentration of surfactant is increased beyond the Critical Micelle Concentration (CMC), micelles form in solution.

Surprisingly, in the present invention, it was found that the addition of non-ionic surfactant at a concentration twice its critical micelle concentration increased the overall yield of the hydrolysis reaction.

It was discovered that the best reagents for hydrolyzing paper into fermentable sugars comprise a blend of cellulase and cellobiase, along with a non-ionic surfactant to facilitate enzyme desorption and improve the yield of the hydrolysis reaction.

In the preferred embodiment of the invention the non-ionic surfactant is added to the paper slurry and allowed to wet the paper fibers for a specific period of time. After the wetting step, the enzyme blend is added and the hydrolysis reaction is initiated. The lag time between adding the non-ionic surfactant and adding the enzyme blend to the paper is at least ten minutes. More preferable, it is at least 30 minutes, and most preferably it is at least one hour.

The foregoing describes the invention in terms of embodiments foreseen by the inventor for which an enabling description was available, notwithstanding that insubstantial modifications of the invention, not presently foreseen, may nonetheless represent equivalents thereto.

### EXAMPLES

The invention is further illustrated in the following examples.

### Preparation of DNS (Dinitrosalicylic Acid) Reagent:

DNS reagent is used as a colorimetric test to quantify reducing sugars such as glucose. Reducing sugars react with 3-5 dinitroslicylic acid (DNS) reagent by reducing the pale yellow colored DNS to the orange-reddish colored, 3-amino, 5-nitrosalicylic acid. The intensity of the color is proportional to the concentration of reducing sugar in solution, therefore, higher concentration of glucose will give darker color.

DNS reagent used in the examples was prepared by dissolving one gram DNS (obtained from Sigma-Aldrich, St. Louis, Mo.) in 50 ml distilled water. 30 grams of sodium potassium tartrate was added to the DNS solution in small batches while stirring the solution on a hot plate. After about 10 minutes, the solution became milky yellow in color. Next 20 ml sodium hydroxide solution (2 Normal) was added while stirring on the hot plate. The solution turned transparent orange. In order to stabilize the color, 0.2 grams sodium bisulphate was added to the mixture. The solution was cooled and stored in an amber colored jar to prevent light induced degradation.

### Preparation of the Glucose Standard Curve:

One gram of glucose (obtained from Sigma-Aldrich, St. Louis, Mo.) was dissolved in 100 ml distilled water to make a 1% glucose solution, which was diluted with distilled water to make 0.1%, 0.2%, 0.3%, and 0.5% glucose solutions. One ml of each solution was added into a test tube along with one ml distilled water and one ml DNS reagent. A blank sample containing 2 ml distilled water and 1 ml DNS reagent was used as control. The test tubes were loosely covered with aluminum foil to minimize evaporation, and all test tubes were placed in a glass beaker containing hot water. The beaker was heated in a boiling water bath for 5 minutes. After cooling the test tubes, a micropipette was used to transfer 200 microliters from each test tube into a well of a flat-bottom 196-well microplate (Corning Life Sciences) and absorbencies were measured at 538 nm using the SpectraMax M2 plate reader (Molecular Devices). The above experiments were repeated five times. The absorbance data for each glucose concentration were averaged and graphed in a straight line of absorption as a function of glucose concentration. Linear regression was used to calculate the equation of the straight line: Absorption=0.9378*glucose concentration (mg/ml)+0.2688. This equation was used to calculate glucose concentration in all examples below.

### Preparation of Paper Slurry:

Buffer solution: 50 mM sodium acetate buffer was prepared by adding 2.05 grams sodium acetate to 500 ml distilled water. The resulting buffer pH was measured at 5 using standard pH paper.
Preparation of the paper slurry: a mixed paper substrate was prepared by combining 4.97 grams newsprint, 4.71 grams white office paper, 1.97 grams magazine paper, and 1.15 grams cardboard. 480 grams of buffer was added to the paper and the mixture was processed in a blender on high speed for 3 minutes until a uniform slurry was made.

### Critical Micelle Concentration (CMC) Measurement:

Different types of surfactants were purchased and their critical micelle concentration (CMC) was measured using the Kruss K11 tensiometer (from Kruss GmbH, Germany) fitted with the Wilhelmy plate. Table 1 provides a list of surfactants used in the experiments and their measured CMC.

More details about surfactant chemical name and purchasing source are given below:

Tween 20: Polysorbate 20, available from Fisher Thermo Scientific, Waltham, Mass.
Tween 80: Polysorbate 80, available from Fisher Thermo Scientific, Waltham, Mass.
Triton X-100: polyoxyethylene octyl phenyl ether, available from Sigma-Aldrich, St. Louis, Mo.
SDS: Sodium Dodecyl Sulfate, available from Sigma-Aldrich, St. Louis, Mo.
CTAC: Cetyl Trimethyl Ammonium Chlorine, available from Clariant, Muttenz, Switzerland.
SLAA: Sodium LauroAmphoAcetate, available from Stobec, Quebec, Canada.

TABLE 1
Surfactants used in the experiment

| Name | Type | MW (g/mol) | CMC (mM) Measured |
|---|---|---|---|
| Tween 20 | Non-ionic | 1228 | 0.094 |
| Tween 80 | Non-ionic | 1310 | 0.008 |
| Triton X-100 | Non-ionic | 628 | 0.18 |
| SDS | Anionic | 288 | 8.3 |
| CTAC | Cationic | 320 | 1.34 |
| SLAA | Zwitterionic | 350 | 3.05 |

Each surfactant was diluted in distilled water to 10% concentration to be used in the following experiments.

### Preparing the Enzyme Blend:

The following two enzymes were mixed to prepare the enzyme blend used in these examples.
1) Cellulase enzyme: Celluclast 1.5L, obtained from Sigma-Aldrich, St. Louis, Mo.

A cellulase enzyme from Trichoderma reesei ATCC 26921, aqueous solution containing 700 units/g.
2) Cellobiase enzyme: Novozyme 188 obtained from Sigma-Aldrich, St. Louis, Mo.

A cellobiase enzyme from Aspergillus niger, 250 units/g.

The blend used in this invention comprises three parts cellulase to one part cellobiase. Taking the active units in each enzyme into consideration, an equal mixture of the two enzyme products results in the desired blend of three parts cellulase to one part cellobiase.

### Examples 1-6 and Comparatives 1C, 2C, and 3C

Each sample was prepared by adding 42 grams of paper slurry to each labeled vial, followed by adding 0.15 grams of the prepared enzyme blend to the sample vials except 1C and 2C as shown in Table 2. Next the relevant surfactants were added to each vial as stated in Table 2 keeping the surfactant concentration at 1.5 times the critical micelle concentration measured from Table 1 for these examples. The samples were placed in a 38° C. water bath for 24 hours.

DNS Assay was used to measure the amount of sugar produced in each example. Out of each sample vial (1C, 2C, 3C, and examples 1-6), 1 ml of solution was removed and placed in a labeled glass test tube. 3 ml distilled water and 2 ml DNS reagent were added to each test tube. The tube was covered loosely with aluminum foil to prevent evaporation and placed in boiling water for 5 minutes to develop the color. The samples were allowed to cool, then 200 μL of each sample were transferred into a flat bottom 96-well plate, and the light absorption was measure at 538 nm using the SpectraMax M2 plate reader. The measured absorption values were used in the glucose absorption curve equation to calculate the concentration of sugar in each sample. The sugar yield reported in Table 2 was calculated based on the weight of paper in each sample. The reported results in Table 2 are the average of five experiments.

TABLE 2
effect of different surfactants on sugar yield.

| | Enzyme | | Sugar |
|---|---|---|---|
| Example | blend | Surfactant | Yield (%) |
| 1C | No enzyme | No surfactant added | 0 |
| 2C | No enzyme | Tween 20 non-ionic surfactant | 0 |
| 3C | 0.15 grams | No surfactant added | 26.15 |
| 1 | 0.15 grams | Cationic | 8.24 |
| 2 | 0.15 grams | Anionic | 27.71 |
| 3 | 0.15 grams | Zwitterionic | 14.36 |
| 4 | 0.15 grams | Tween 20 non-ionic surfactant | 36.62 |
| 5 | 0.15 grams | Tween 80 non-ionic surfactant | 35.04 |
| 6 | 0.15 grams | Triton X-100 none ionic surfactant | 36.13 |

The results of comparative examples 1C and 2C indicate that no sugar was produced without the presence of enzyme and that Tween 20 does not result in measurable light absorption in the sample. Comparative example 3C shows that without surfactant the cellulose to sugar conversion is about 26%. Anionic surfactant does not have significant effect on sugar yield, while cationic and zwitterionic surfactants had negative effect on the hydrolysis reaction resulting in significant reduction in sugar yield. The addition of non-ionic surfactant increased the sugar yield significantly, and all three non-ionic surfactants resulted in similar levels of improvement, about 45% increase in sugar yield.

### Examples 7-29: Non-Ionic Surfactant Concentration Effect

The effect of non-ionic surfactant concentration was examined using three different nonionic surfactants: Tween 20, Tween 80 and Triton X-100.

To prepare each example 42 grams of paper slurry was added to each labeled vial, followed by adding 0.15 grams of the prepared enzyme blend along with the relevant concentration of each surfactant as stated in Table 3. The samples were placed in a 38° C. water bath for 24 hours.

DNS Assay was used to measure the amount of sugar produced in each example. Out of each sample vial 1 ml was removed and placed in a labelled test tube. 3 ml distilled water and 2 ml DNS reagent were added. Each test tube was covered loosely with aluminum foil to prevent evaporation and place in boiling water for 5 minutes to develop the color. The samples were allowed to cool, then 200 μL of each sample were transferred into a flat bottom 96-well plate, and the light absorption was measure at 538 nm using the SpectraMax M2 plate reader. The measured absorption value was used in the glucose absorption curve equation to calculate the concentration of sugar in each sample. The sugar yield reported in Table 3 was calculated based on the weight of paper in each sample. The reported results in Table 3 are the average of five experiments.

TABLE 3
Effect of non-ionic surfactant type and concentration.

| | | Surfactant | |
|---|---|---|---|
| Example | Surfactant | Concentration (mM) | Sugar Yield (%) |
| 7 | none | 0 | 26.51 |
| 8 | Tween 20 | 0.04 | 29.53 |
| 9 | Tween 20 | 0.07 | 32.14 |
| 10 | Tween 20 | 0.11 | 34.84 |
| 11 | Tween 20 | 0.14 | 38.12 |
| 12 | Tween 20 | 0.18 | 39.50 |
| 13 | Tween 20 | 0.22 | 37.91 |
| 14 | Tween 20 | 0.29 | 29.64 |
| 15 | Tween 20 | 0.36 | 23.34 |
| 16 | Triton X-100 | 0.07 | 29.22 |
| 17 | Triton X-100 | 0.14 | 34.03 |
| 18 | Triton X-100 | 0.22 | 36.21 |
| 19 | Triton X-100 | 0.29 | 40.94 |
| 20 | Triton X-100 | 0.36 | 42.13 |
| 21 | Triton X-100 | 0.43 | 41.52 |
| 22 | Triton X-100 | 0.50 | 33.75 |
| 23 | Triton X-100 | 0.57 | 27.40 |
| 24 | Tween 80 | 0.01 | 36.88 |
| 25 | Tween 80 | 0.02 | 40.19 |
| 26 | Tween 80 | 0.036 | 32.44 |
| 27 | Tween 80 | 0.07 | 27.65 |
| 28 | Tween 80 | 0.11 | 24.08 |
| 29 | Tween 80 | 0.14 | 21.75 |

Sugar yield increased with increasing surfactant concentration up to about twice the critical micelle concentration of each surfactant, which is 0.094 mM for Tween 20, 0.18 mM for Triton X-100 and 0.008 for Tween 80 as reported in Table 1. Higher concentration of each surfactant resulted in decrease in yield as shown in Table 3. The same trend was observed for all three non-ionic surfactants eventhough they have different CMC values.

Not wishing to be bound by theory, it is believed that the observed decrease in sugar yield as surfactant concentration is farther increased beyond twice the CMC value maybe due to the enzyme molecules sequestered inside the surfactant micelles that are formed from excess surfactant in soluiton. Removing enzyme molecules by sequestering inside the surfactant micelles results in decrease in reaction rate, lowering the measured yield of the hydrolysis reaction.

### Example 30: Effect of Lag Time Between Surfactant and Enzyme Addition

Example 12 was repeated except a lag time was introduced between surfactant addition and enzyme blend addition. To prepare example 30, 42 grams of paper slurry was added to a labeled vial, followed by adding Tween 20 non-ionic surfactant to make a solution of 0.18 mM surfactant concentration. The mixture was allowed to equilibrate at 38° C. for one hour, then 0.15 grams of the enzyme blend was added to the vial, and the sample was placed in a 38° C. water bath for 24 hours.

DNS Assay was used to measure the amount of sugar produced. Out of the sample vial 1 ml was removed and placed in a test tube. 3 ml distilled water and 2 ml DNS reagent were added. The test tube was covered loosely with aluminum foil to prevent evaporation and place in boiling water for 5 minutes to develop the color. The sample was allowed to cool, then 200 μL were transferred into a flat bottom 96-well plate, and the light absorption was measure at 538 nm using the SpectraMax M2 plate reader. The absorption value was used in the glucose absorption curve equation to calculate the concentration of sugar in the sample. The experiment was repeated five times and the average value for sugar yield was calculated from the glucose absorption curve equation to be 46%, significantly higher than the 39% obtained in example 12.

I claim:

1. A method for producing fermentable sugars from paper, comprising:
a. Treating an aqueous mixture of paper with non-ionic surfactant;
b. Adding an enzyme blend to the mixture from step a;
c. Incubating the mixture from step b at a temperature ranging from 30° C. to 50° C. to make a fermentable aqueous mixture comprising sugars;
d. Filtering the mixture to remove paper residue;
wherein the enzyme blend from step (b) comprises a blend of cellulase and cellobiase.

2. The method of claim 1 wherein the enzyme blend comprises three parts cellulase and one part cellobiase.

3. The method of claim 1 wherein the enzyme blend concentration is at least 20% by weight of the paper.

4. The method of claim 1 wherein the enzyme blend concentration is at least 10% by weight of the paper.

5. The method of claim 1 wherein the non-ionic surfactant concentration is at least equal to the critical micelle concentration of the surfactant.

6. The method of claim 1 wherein the non-ionic surfactant concentration is greater than its critical micelle concentration.

7. The method of claim 1 wherein the non-ionic surfactant concentration is at least 1.5 times the critical micelle concentration of the surfactant.

8. The method of claim 1 wherein the non-ionic surfactant concentration is at least twice the critical micelle concentration of the surfactant.

9. The method of claim 1 where the time between steps a and b is at least one hour.

10. The method of claim 1 where the pH of the surfactant treated paper is adjusted to be between 5.5 and 6.5.

11. A method to convert waste paper to fermentable sugar comprising:
a. Mixing waste paper with a buffer solution using high shear mixing method;
b. Adding non-ionic surfactant to the mixture from step a;
c. Adding an enzyme blend comprising cellulase and cellobiase to the mixture from step b;
d. Incubating the mixture from step c at a temperature ranging from 30° C. to 50° C.
e. Filtering the mixture to remove paper residue;
wherein the enzyme blend from step (b) comprises a blend of cellulase and cellobiase.

12. The method of claim 11 wherein the enzyme blend comprises three parts cellulase and one part cellobiase.

13. The method of claim 11 wherein the enzyme blend concentration is at least 20% by weight of the paper.

14. The method of claim 11 wherein the enzyme blend concentration is at least 10% by weight of the paper.

15. The method of claim 1 wherein the non-ionic surfactant concentration is at least equal to the critical micelle concentration of the surfactant.

16. The method of claim 11 wherein the non-ionic surfactant concentration is greater than its critical micelle concentration.

17. The method of claim 11 wherein the non-ionic surfactant concentration is at least 1.5 times the critical micelle concentration of the surfactant.

18. The method of claim 11 wherein the non-ionic surfactant concentration is at least twice the critical micelle concentration of the surfactant.

19. The method of claim 11 where the time between steps b and c is at least one hour.

20. The method of claim 11 where the pH of the surfactant treated paper is adjusted to be between 5.5 and 6.5.
